(19) United States
(12) Patent Application Publication
(10) Pub. No.: US 2022/0296637 A1
(43) Pub. Date: Sep. 22, 2022

(54) SODIUM THIOSULFATE GEL FOR PREVENTING OR REDUCING HEARING LOSS

(71) Applicant: STICHTING HET NEDERLANDS KANKER INSTITUUT-ANTONI VAN LEEUWENHOEK ZIEKENHUIS, AMSTERDAM (NL)

(72) Inventors: Alfonsus Jacobus Maria BALM, AMSTERDAM (NL); Jacob Hendrik BEIJNEN, AMSTERDAM (NL); Bastiaan NUIJEN, AMSTERDAM (NL); Johannes Henricus Matthias SCHELLENS, AMSTERDAM (NL); Charlotte Louise ZUUR, AMSTERDAM (NL)

(21) Appl. No.: 17/619,076

(22) PCT Filed: Jun. 19, 2020
(86) PCT No.: PCT/NL2020/050391

§ 371 Date: Dec. 14, 2021

(30) Foreign Application Priority Data
Jun. 19, 2019 (NL) 2023344
May 8, 2020 (NL) 2025531

(51) Int. Cl. A61K 33/04; A61K 9/06; A61K 9/00; A61K 31/555; A61K 33/243; A61P 27/16

(52) U.S. Cl. CPC A61K 33/04; A61K 9/06; A61K 9/0019; A61K 9/0046; A61K 31/555; A61K 33/243; A61P 27/16

(57) ABSTRACT

Described herein is sodium thiosulfate (STS) for use in preventing or reducing hearing loss in patients receiving platinum-based drugs, for example as chemotherapy to treat cancer. The invention further relates to STS gel compositions suitable for administering to the middle ear of a patient in need thereof, particularly a patient receiving platinum-based drugs.

### FIELD OF THE INVENTION

The invention relates to sodium thiosulfate (STS) for use in the reduction of platinum-based drug induced hearing loss. The invention also relates to the use of a platinum-based drug, combined with the administration of STS, for use in the reduction of platinum-based drug induced hearing loss. The invention further relates to a kit of parts comprising an ampoule and/or injections needle comprising STS.

### INTRODUCTION

Platinum-based drugs (platins) are chemotherapeutic agents used to treat cancer. Platins are for example used in the treatment of amongst others paediatric, thoracic, head and neck, skin, urologic, gastro-enterologic and gynaecologic solid tumours. Platins initiate cell death via intra- and inter-strand crosslinking between the platinum atoms and the tumour's DNA purine bases. The number of cycles and the total dose of platins that can be administered is limited due to cumulative toxicity that consists of neuropathy, nephropathy, and ototoxicity. Nephrotoxicity can largely be prevented by intravenous hyper-hydration prior to and after infusion of cisplatin. However, for ototoxicity and neuropathy no suitable preventive or curative strategies are available.

Platinum-based drug induced hearing loss, such as cisplatin-induced hearing loss (CIHL), occurs in 75-80% of the cisplatin-treated patients, although reported incidences vary widely amongst studies due to heterogeneity in the given cisplatin dose and in the definitions used for ototoxicity. CIHL is dose-dependent and starts shortly after treatment (i.e. days). It is characterized by symmetric, bilateral, and irreversible sensorineural hearing loss (SNHL). For example, for cisplatin it is known that it destructs the hair cells (HCs) within the organ of Corti. The outer HCs located at the basal cochlear turns are first affected, leading to SNHL at the ultrahigh frequencies. After succeeding doses, cisplatin affects the outer HCs located at the apical cochlear windings and SNHL progresses to the lower frequencies. The inner HCs, spiral ganglion, and stria vascularis may also be damaged by cisplatin. Other platinum-based drugs, for example carboplatin and oxaliplatin, were found to be ototoxic through the same mechanism.

At the molecular level, the pathophysiology of platinum-based drug induced hearing loss is believed to consist of the formation of toxic levels of reactive oxygen species (ROS) and the depletion of cochlear otoprotective antioxidants. Distinct cochlear antioxidants protect the cochlea from ototoxic stress, but seem to be unable to deal with the cochlear damage by platinum-based drugs. Therefore, the use of antioxidants that aim to reduce the production and/or activity of ROS has been proposed. Various antioxidants, including sodium thiosulfate (STS) have been studied for their otoprotective effects. STS does not only scavenge ROS produced by platinum-based drugs, but it also inactivates platinum-based drugs by direct binding to the active form of the drug.

Recently, two phase III trials on the prevention of CIHL in children showed promising otoprotective effects of intravenous STS next to/administered together with standardized cisplatin therapy: the incidence of CIHL was remarkably lower in children that were treated with cisplatin plus intravenously administered STS compared with cisplatin alone. Brock et al. showed in 109 children that the incidence was 48% lower in the STS-treated group (relative risk, 52%; 95% confidence interval, 0.33-0.81, p=0.002). Similarly, Freyer et al. showed that CIHL was seen in 29% of the 61 patients treated with STS and in 56% of the 64 patients treated with cisplatin alone (p=0.00022). However, a restriction of the use of systemic STS is the potential interference with the anti-tumour activity of cisplatin and the side effects of systemic STS. Accordingly, in the study of Freyer et al., children with disseminated disease had a significantly lower overall survival when treated with additional intravenous STS (45%) compared with the children that were treated with cisplatin alone (84%) (p=0.009). Furthermore, Brock et al. reported adverse events that were likely attributed to intravenous STS administration, including tumour progression and grade 3 infection, neutropenia, electrolyte disorders and anaemia.

As an alternative approach, several preclinical proof-of-principle studies showed that the transtympanic application of antioxidants is safe and feasible. In guinea pigs, higher perilymph STS concentrations were achieved when applied with transtympanic injections compared with an intravenous infusion.

However, recently, Rolland et al. evaluated the transtympanic application of STS gel in 13 human patients treated with concomitant radiotherapy and cisplatin for HNSCC. They showed that the averaged hearing loss was 1.3 dB less in the STS-treated ears compared to the untreated ears at frequencies from 3 to 10 kHz, and were unable to demonstrate a statistically significant and/or clinically relevant effect of the STS gel.

Consequently there is still an unmet need to provide for methods to prevent or reduce hearing loss induced by platinum-based drugs in humans. The invention therefore aims to overcome these problems by the uses and compositions as described in the appended claims.

### SUMMARY OF THE INVENTION

In a first aspect the invention relates to sodium thiosulfate (STS) for use in preventing or reducing hearing loss induced by a platinum-based drug wherein, STS is administered to the middle ear of a human subject between 8 hours before and 8 hours after the platinum-based drug is administered to said human subject.

In a further aspect the invention relates to platinum-based drug for use in preventing or reducing hearing loss induced by the platinum-based drug wherein STS is administered to the middle ear of a human subject between 8 hours before and 8 hours after the platinum-based drug is administered to said human subject.

In a further aspect the invention relates to a composition, preferably a gel composition, comprising STS and/or a composition comprising a platinum-based drug for use in preventing or reducing hearing loss induced by the platinum-based drug wherein STS is administered to the middle ear of a human subject between 8 hours before and 8 hours after the platinum-based drug is administered to said human subject.

In a further aspect the invention relates to a kit of parts comprising:
a container, preferably an ampoule, a syringe and/or an injection needle, comprising a pharmaceutical acceptable composition, preferably a gel composition, preferably a hyaluronate-based gel composition, comprising STS, wherein said container, preferably an ampoule, a syringe and/or an injection needle is for administration of between 0.05 and 5.0 ml of said composition to the middle ear of a human subject, preferably wherein the said container, preferably an ampoule, a syringe and/or an injection needle is for single-use; and/or
a first container, preferably an ampoule, comprising an aqueous solution comprising STS and a second container, preferably a syringe and/or an injection needle, comprising a gel (herein also referred to as pre-gel), preferably a gel with a composition consisting of: an aqueous solution of 1.0% sodium hyaluronate (preferably in a PBS buffer as disclosed herein).

In a further aspect the invention relates to a method of preventing or reducing hearing loss induced by a platinum-based drug in a human subject, the method comprising that STS is administered to the middle ear of a human subject between 8 hours before and 8 hours after the platinum-based drug is administered to said human subject.

### FIGURE LEGENDS

FIG. 1: Trial profile

FIG. 2: Audiometric results of the 12 patients in both arm A and B. The curves show hearing at baseline, directly after the last cycle of cisplatin (post-treatment) and at follow up. Up: Pure tone audiometry, mean of the thresholds in dB HL. Down: Ultrahigh frequency audiometry, mean of the air conduction thresholds in dB SPL. In some patients the thresholds at 8 kHz were not available for ultrahigh frequency audiometry. These values have been converted from the threshold measured with regular pure-tone audiometry into dB SPL following ISO 389-1.

FIG. 3: Audiometric results of the 4 patients that did not develop ototoxicity (defined as a shift of ≥10 dB at ΔPTA 1-2-4 kHz (ΔPTA 1-2-4 kHz=PTA 1-2-4 kHz directly post-treatment minus PTA 1-2-4 kHz at baseline) and/or ΔPTA 8-10-12.5 kHz (ΔPTA 8-10-12.5 kHz=PTA 8-10-12.5 kHz directly post-treatment minus PTA 8-10-12.5 kHz at baseline). The curves show hearing at baseline, directly after the last cycle of cisplatin (post-treatment) and at follow up. FIG. 2A: Pure tone audiometry, mean of the thresholds in dB HL. FIG. 2B: Ultrahigh frequency audiometry, mean of the air conduction thresholds in dB SPL. In some patients the thresholds at 8 kHz were not available for ultrahigh frequency audiometry. These values have been converted from the threshold measured with regular pure-tone audiometry into dB SPL following ISO 389-1.

FIG. 4: Audiometric results of the 4 patients that suffered ototoxicity and responded to the transtympanic (also known as ‘intra-tympanic’) sodium thiosulfate (STS) injection to the middle ear. Response is defined as patients with ototoxicity in which ΔPTA 8-10-12.5 kHz in the untreated ear exceeds ΔPTA 8-10-12.5 kHz in the STS-ear with ≥10 dB (ΔPTA=PTA directly post-treatment minus baseline PTA). The curves show hearing at baseline, directly after the last cycle of cisplatin (post-treatment) and at follow up. FIG. 4A: Pure tone audiometry, mean of the thresholds in dB HL. FIG. 4B: Ultrahigh frequency audiometry, mean of the air conduction thresholds in dB SPL. In some patients the thresholds at 8 kHz were not available for ultrahigh frequency audiometry. These values have been converted from the threshold measured with regular pure-tone audiometry into dB SPL following ISO 389-1.

FIG. 5: Audiometric results of the 4 patients that suffered ototoxicity and did not respond to the transtympanic sodium thiosulfate (STS) injection. Response is defined as patients with ototoxicity in which ΔPTA 1-2-4 kHz in the untreated ear exceeds ΔPTA 1-2-4 kHz in the STS ear and/or ΔPTA 8-10-12.5 kHz in the untreated ear exceeds ΔPTA 8-10-12.5 kHz in the STS-ear with ≥10 dB (ΔPTA=PTA directly post-treatment minus baseline PTA). The curves show hearing at baseline, directly after the last cycle of cisplatin (post-treatment) and at follow up. FIG. 5A: Pure tone audiometry, mean of the thresholds in dB HL. FIG. 5B: Ultrahigh frequency audiometry, mean of the air conduction thresholds in dB SPL. In some patients the thresholds at 8 kHz were not available for ultrahigh frequency audiometry. These values have been converted from the threshold measured with regular pure-tone audiometry into dB SPL following ISO 389-1.

FIG. 6: Concentration-time curves of total platinum and unbound platinum. The curves show the mean concentrations (in nanogram (ng) per milliliter (ml)) of platinum for 8 patients treated with 75 mg/m² of cisplatin (x) and 3 patients treated with 100 mg/m² of cisplatin (o). SOI=start of infusion, EOI=end of infusion.

### DEFINITIONS

The term “platinum-based drug” as used herein refers to a drug wherein the active compound comprises a platinum complex. Such drugs are coordination complexes of platinum. When used to treat cancer (chemotherapy) such drug are referred to as platinum-based anticancer drugs, or platinum-based antineoplastics. When used as chemotherapy to treat cancer, exemplary drugs include cisplatin, oxaliplatin, carboplatin and nedaplatin, but several others have been proposed or are under development. Therefore platinum-based drug when used herein may refer to any complex of platinum which is used to treat a patient, preferably to treat cancer in a patient. Non limiting examples of platinum-based drugs are cisplatin, carboplatin, oxaliplatin, nedaplatin, triplatin tetranitrate, phenanthriplatin, picoplatin and satraplatin.

Ototoxicity, is known to the skilled person to be the property of being toxic to the ear (oto-), specifically the cochlea or auditory nerve and sometimes the vestibular system, for example, as a side effect of a drug. The effects of ototoxicity can be reversible and temporary, or irreversible and permanent. Symptoms of ototoxicity include partial or profound hearing loss and/or tinnitus. Hearing loss when used herein refers to an increase in the threshold volume at which sound of a certain frequency or frequency range can be perceived. Therefore, reducing hearing loss when used herein refers to a lower increase or absence of said threshold compared to the situation where the hearing-loss occurred. Alternatively preventing hearing loss refers to an absence of increase in the threshold volume at which sound of a certain frequency or frequency range can be perceived when compared with the situation that hearing-loss, and therefore such increase in the threshold volume at which sound of a certain frequency or frequency range can be perceived, did occur.

When used herein, platinum-based drug induced hearing loss refers to the hearing-loss induced by a platinum-based drug as defined herein. It is well known in the field that platinum-based drugs such as cisplatin, carboplatin and oxaliplatin are ototoxic and may result in hearing-loss. Platinum-based drug induced hearing loss is characterized by symmetrical (equal in both ears of one patient) and permanent sensorineural hearing loss. The platinum-based drug induced hearing loss may be accompanied by tinnitus, or may be without tinnitus.

When referring to platinum-based drug administration, “dosage”, when used herein means the amount of platinum-based drug administered to a patient in a single event. Since platinum-based drugs are typically administered intravenously, a single event means the cumulative amount of platinum-based drug administered, typically intravenously, typically over the period of one or more hours. When multiple platinum-based drugs are provided simultaneously the dosage refers to the cumulative amount of platinum-based drugs provided in a single event. When subsequent administration takes places of the same or different platinum-based drugs within a short time span, e.g. within the span of 24, or 12 hours or less, such subsequent administrations should be counted as a single event, and thus the dosage is the cumulative amount of these subsequent dosages which are counted as single event.

Platinum-based drugs are typically provided intravenously and therefore are administered over a time course, for example over the time of one to four hours. Therefore, when referred herein to the time wherein STS is to be administered before administration of the platinum-based drug, such time refers to the onset (start) of administration of said platinum-based drug, wherein said onset is the introduction of the platinum-based drug in the subject (e.g. I.V. (intravenous) system. Therefore, when referred herein to the time wherein STS is to be administered after administration of the platinum-based drug, such time refers to the time from the termination (stop) of administration of said platinum-based drug, wherein said termination is the time point the platinum-based drug has all been introduced intravenously to the subject or when the platinum-based drug is disconnected from the I.V. system. When the administration event comprises multiple successive administrations within a short time span, the onset (start) refers to the onset (start) of the first of the series of administrations and the termination (stop) refers to the termination (stop) of the last of the series of administrations. For example, STS may be provided to the subject up to 8 hours before the start of the treatment with this platinum-based drug, and/or during the treatment with the platinum-based drug, and/or up to 8 hours after the treatment with the platinum-based drug has ended.

When used herein, the middle ear is the portion of the ear internal to the eardrum, and external to the oval and round windows of the inner ear. The middle ear can be reached via the ear drum or via the Eustachian tube. Therefore, administration to the middle ear, when used herein, refers to administration of a substance to the cavity of the ear internal to the eardrum and external to the oval and round windows. Administration of the drug to the middle ear can take place via the ear drum (so-called ‘transtympanically’ or ‘intra-tympanically’ administration of the drug) or via the Eustachian tube.

When referred herein, administration of a platinum-based drug refers to administering of a platinum-based drug, for example cisplatin, carboplatin and oxaliplatin to a human subject (patient). Typically, such drugs are administered intravenously.

When used herein transtympanical refers to accessing or entering through the eardrum. Therefore, transtympanical administration refers to the administration through the eardrum. For example, such administration can be performed with a suitable syringe with a suitable needle or canula.

Intratympanical refers to accessing or entering into the middle ear. Therefore, intratympanical administration refers to the administration into the middle ear. Intratympanical administration may be performed through the eardrum, but may also be done in a different way, for example through the Eustachian tube. Therefore, transtympanical administration is a form (species) of intratympanical administration (genus) when used herein.

A gel composition, when used herein refers to a solid jelly-like soft material. Gels are generally regarded as a non-fluid colloidal network or polymer network that is expanded throughout its whole volume by a fluid. Preferably the gel composition according to the invention is a hydrogel, meaning a gel in which the swelling agent is water.

When used herein, the viscosity refers to the intrinsic viscosity (m³/kg) as determined according to the current European Pharmacopoeia (Ph Eur 2.2.9/monograph of sodium hyaluronate) at both 25° C. as well as 37° C. Preferably the intrinsic viscosity is determined at 25° C. or at 37° C. More preferably the intrinsic viscosity is determined at 37° C. Therefore, when referring to a liquid or gel having a particular intrinsic viscosity, for example the intrinsic viscosity of a 0.5% sodium hyaluronate solution, said intrinsic viscosity is the intrinsic viscosity that is determined at 25° C. or at 37° C., preferably at 37° C. Intrinsic viscosity is a measure of a solute's contribution to the viscosity of a solution. Preferably the intrinsic viscosity at this temperature (25° C. or 37° C., preferably at 37° C.) is similar as the intrinsic viscosity of a gel which comprises 0.1 M sodium thiosulfate and 0.5% sodium hyaluronate in phosphate buffered saline pH7.4 (PBS). PBS typically comprises 137 mmol/L NaCl, 2.7 mmol/L KCl, 10 mmol/L Na₂HPO₄ and 1.8 mmol/L KH₂PO₄ at the same temperature. A similar intrinsic viscosity means having an intrinsic viscosity of between 75% and 150% of the intrinsic viscosity of a solution which comprises 0.1 M sodium thiosulfate and 0.5% sodium hyaluronate in phosphate buffered saline pH7.4 (PBS). More preferably the intrinsic viscosity is between 80% and 140%, 85% and 130%, 90% and 120% or 95% and 110% of a solution which comprises 0.1 M sodium thiosulfate and 0.5% sodium hyaluronate in phosphate buffered saline pH7.4 (PBS).

Hyaluronate solutions show pseudoplastic behavior which means that they become less viscous with higher shear due to alignment of the polymer molecules, which makes it particularly suitable for injection through a needle. Therefore in a preferred embodiment the gel has pseudoplastic behavior.

When used herein, a grommet refers to a tympanostomy tube, also known as myringotomy tube, and is a small tube inserted into the eardrum in order to keep the middle ear aerated for a prolonged period of time. The grommet may be used for transtympanical administration, meaning that a needle is inserted in the middle ear through the grommet. Alternatively, the grommet may serve to equalize the pressure in the middle ear when the transtympanical administration is performed through the eardrum itself (by piercing the eardrum). It is to be noted that the presence of a grommet in the ear is not required for the uses and methods described herein.

Pre-gel, when used herein refers to a gel-like material that does not yet comprise STS, and which has an intrinsic viscosity which is chosen such that when mixed at a pre-determined mixing ratio with an STS comprising solution, the resulting gel provides as intrinsic viscosity (at 25 or 37 degrees Celsius, preferably at 37 degrees Celcius) that is suitable for use in the treatment as disclosed herein, e.g. is within the viscosity-range as defined herein. For example, the pre-gel has an intrinsic viscosity which is such that upon mixing 1:1 with the STS solution the resulting intrinsic viscosity is within the desired range. Similarly, the STS solution has a higher concentration than the desired concentration for administration, such that upon mixing the STS solution with the pre-gel, the resulting concentration is as desired for use in a patient. The skilled person is aware that depending on the intended mixing ratios of the pre-gel and the STS solution, the intrinsic viscosity of the pre-gel and the concentration of STS in the STS solution need to be varied accordingly. Preferred mixing ratios may be 5:1, 4:1. 3:1, 2:1, 1:1, 1:2, 1:3, 1:4 and 1:5, but other suitable mixing ratios of the pre-gel and STS-solution may be chosen as well.

In this document and in its claims, the verb “to comprise” and its conjugations is used in its non-limiting sense to mean that items following the word are included, but items not specifically mentioned are not excluded. In addition, the verb “to consist of may be replaced by “to consist essentially of” meaning that a composition may comprise additional component(s) than the ones specifically identified, or a method may comprise additional step(s) than the ones specifically identified, said additional component(s) or additional step(s) not altering the unique characteristics of the invention.

In addition, reference to an element by the indefinite article “a” or “an” does not exclude the possibility that more than one of the elements is present, unless the context clearly requires that there be one and only one of the elements. The indefinite article “a” or “an” thus usually means “at least one”.

### DETAILED DESCRIPTION

The present invention aims to prevent or reduce platinum-based drug induced hearing loss, therefore in a first embodiment the invention relates to sodium thiosulfate (STS) for use in preventing or reducing hearing loss induced by a platinum-based drug, wherein STS is administered to the middle ear of a human subject between 8 hours before and 8 hours after the platinum-based drug is administered to said human subject.

Also provided is for sodium thiosulfate (STS) for use the treatment of cancer, wherein STS is administered to the middle ear of a human subject between 8 hours before and 8 hours after a platinum-based drug is administered to said human subject.

Platinum-based drugs (platins) are chemotherapy medication used to treat a number of cancers. A major drawback is the many side effects caused by toxicity of platins to the patient. One such side effect is hearing loss. It has been proposed that sodium thiosulfate (STS), which inhibits platins, may be used to counteract platinum-based drug induced hearing loss. Unfortunately, systemic administration of STS result in an increase in mortality in patients, likely due to the blocking of platins. To solve this it was proposed to administer an STS containing gel to the middle ear. A study described in Rolland et al. however failed to demonstrate significant (neither statistically significant, nor clinically relevant) improvements using such methods. In this study, STS was administered 24 hours prior to administration of a platinum-based drug. It has now surprisingly been found that good results can be obtained by administering STS 8 hours prior to up to 8 hours after the administration of the platinum-based drug. The results described herein demonstrate clinically relevant efficacy.

Without wishing to be bound by theory, it is theorized that administration of STS in a time window of 8 hours before and 8 hours after administration of the platinum-based drug, allows for optimal presence in the middle ear relative to the presence of the platinum-based drug. Providing STS outside the time window is believed to provide for suboptimal STS levels relative to the platinum-based drug and thus no or only limited prevention or reduction of hearing loss. It is theorized that the STS administered to the middle ear will have its efficacy at the level of the inner ear, putatively via diffusion though the round window. It is theorized that STS inhibits ROS formation (in the inner ear) through binding of platinum-based drugs.

Therefore, the present invention further relates to a method of treatment wherein STS is administered to a human patient between 8 hours prior to up to 8 hours after the administration of a platinum-based drug to said patient. Preferably said method of treatment is to reduce platinum-based rug induced hearing loss.

In an embodiment of the invention, the platinum-based drug is selected from the group consisting of cisplatin, carboplatin, oxaliplatin, nedaplatin, picoplatin, triplatin tetranitrate, satraplatin and phenanthriplatin, or a mixture thereof, preferably wherein the platinum-based drug is selected from the group consisting of cisplatin, carboplatin, oxaliplatin and nedaplatin. The skilled person is aware that ototoxicity has been demonstrated for several different platinum-based drugs, therefore the present invention is not limited to a single platinum-based drug but encompasses any platinum-based drug. Non-limiting examples of platinum-based drugs are cisplatin, carboplatin, oxaliplatin, nedaplatin, picoplatin, triplatin tetranitrate, satraplatin and phenanthriplatin. Preferred examples are cisplatin, carboplatin, oxaliplatin and nedaplatin, as these platinum-based drugs have been approved to the market and have been demonstrated to be ototoxic as such.

Cisplatin, or Pt(NH₃)₂Cl₂, is also known to the skilled person as cisplatinum, platamin, neoplatin, cismaplat or cis-diaminedichloroplatinum(II), and is commercially available as Platinol®. Carboplatin, sold under the trade name Paraplatin® among others, is also known to the skilled person as cis-diamine(cyclobutane-1,1-dicarboxylate-O,O′)platinum(II). Oxaliplatin, sold under the brand name Eloxatin®, is also known to the skilled person as [(1R,2R)-cyclohexane-1,2-diamine](ethanedioato-O,O′)platinum(II). Nedaplatin, marketed under the name Aqupla, is known to the skilled person as Diammine[(hydroxy-KO)acetato(2-)-κO]platinum. Picoplatin is known to the skilled person as a complex of azane; 2-methylpyridine; platinum(2+); dichloride. Triplatin tetranitrate is also known as BBR3464. Satraplatin is also known as JM216, BMY 45594, BMS 182751 and (OC-6-43)-bis(acetato)amminedichlorocyclohexylamine platinum(IV). Phenanthriplatin is also known as cis-[Pt(NH₃)₂-(phenanthridine)Cl]NO₃.

In an embodiment, the STS is comprised in a pharmaceutically acceptable composition, preferably a gel composition (e.g. a hydrogel, not or only to a limited extent showing flow). Pharmaceutically acceptable compositions which are suitable for administration to the middle ear are known to the skilled person, for example methylcellulose based gel compositions.

STS is preferably administered as part of a gel composition. Without wishing to be bound be theory, it is theorized that one advantage of using a gel composition, in combination with e.g. the timing of providing the STS to the human subject) is that the composition stays longer in the middle ear, compared with a composition with a very low viscosity (such as a non-gel like liquid). A low viscous fluid may quickly drain away from the middle ear through the Eustachian tube, which may result in lower STS concentrations in the relevant parts of the ear to exert its protective effect. Therefore, a gel is preferred with a viscosity which is such that the liquid does not drain away quickly, but can still be administered, for example using a suitable syringe. The skilled person is aware how to choose a suitable viscosity, and how to prepare a suitable gel having said viscosity.

The skilled person is aware how to prepare a gel composition suitable for administering to the middle ear. For example a gel may be prepared from sodium hyaluronate, however the skilled person is aware of other gels that are suitable for this purpose. For example, suitable gels could also be prepared from methylcellulose. In a beneficial embodiment a gel is prepared from sodium hyaluronate with a concentration ranging between 0.2 and 1.0% sodium hyaluronate, more preferably between 0.3% and 0.8%, more preferably between 0.4% and 0.6% most preferably around 0.5% sodium hyaluronate, as described herein, preferably in PBS as described herein. Alternatively, a gel may prepared with a different gelling agent but with a corresponding intrinsic viscosity compared to the intrinsic viscosity of the sodium hyaluronate gels, therefore, in a preferred embodiment the gel has an intrinsic viscosity which is comparable as the intrinsic viscosity of a gel prepared from sodium hyaluronate with a concentration ranging between 0.2 and 1.0% sodium hyaluronate, more preferably between 0.3% and 0.8%, more preferably between 0.4% and 0.6% most preferably around 0.5% sodium hyaluronate.

The skilled person is further aware that additional adjuvants may be provided to the composition, such as but not limited to, salts, buffers or preservatives.

In an embodiment of the invention the STS is administered by transtympanic or intratympanic injection. It is particularly beneficial to administer the STS to the middle ear of a patient, therefore said administration of STS is preferably done transtympanically or intratympanically. Alternatively it is also envisioned that the STS can be administered to the middle ear via the Eustachian tube.

When used herein transtympanic refers to through the eardrum. Transtympanic administration may be done for example using a needle. The administration may be done by any opening present in the eardrum, for example by placing a grommet in the eardrum and inserting the needle in the grommet, or the administration may be done by directly piercing the eardrum with the needle.

The eardrum, also called the tympanic membrane or myringa, is a thin, cone-shaped membrane that separates the external ear from the middle ear. Therefore, the object of transtympanical administration is to deliver a substance, in the case of the present invention STS, for example STS comprising gel composition, to the middle ear, through the eardrum/PCT.

When used herein intratympanic refers to into the middle ear, therefore intratympanic administration encompasses transtympanic administration, through the eardrum, but also includes other modes of delivery of a substance to the middle ear which are not transtympanical. An example of intratympanical administration that is not transtympanical administration, is administration in the middle ear through the Eustachian tube.

In an embodiment of the invention the dosage of the platinum-based drug is 20 mg/m² or more, 40 mg/m² or more, 75 mg/m² or more, preferably 100 mg/m² or more. Hearing loss in platinum-based drug treated patients is particularly observed when higher dosages of platinum-based drug are administered, therefore a particular attractive embodiment of the invention is to administer STS, for example STS comprising gel composition, before or after a high dosage of platinum-based drug to said patient, preferably a dosage of at least 20 mg/m², more preferably at least 25, 30, 35, 40, 45, 50, 55, 60, 65, 70, 75, 80, 85, 90 or 95 mg/m², even more preferably at least 100 mg/m² or more, 150 mg/m² or more or 200 mg/m² or more, is provided. When multiple platinum-based drugs are administered simultaneously, the dosage refers to the cumulative amount of platinum-based drugs administered in a single event as defined herein. It is further envisioned that STS is administered in case increasing doses of a platinum-based drug are administered. For example in a first round 30 mg/m² of a platinum-based drug is administered, in a second round 50 mg/m² of a platinum-based drug is administered and in a third round 80 mg/m² of a platinum-based drug is administered, while for each round also STS is administered between 8 hours before or after the administration of the platinum-based drug.

The dosage of platinum-based drug can be determined by the skilled person. It is to be understood that in case multiple administrations of platinum-based drugs are provided to a patient which are counted as separate events (as defined above), that preferably each administration is preceded by the administration of STS according to the invention, more preferably each administration of at least 40 mg/m² of platinum-based is preceded or followed by the administration of STS according to the invention.

In an embodiment of the invention, when the platinum-based drug is cisplatin the dosage of cisplatin administered to said patient is 20 mg/m² or more, 40 mg/m² or more, 75 mg/m² or more, preferably 100 mg/m² or more; when the platinum-based drug is carboplatin the dosage of carboplatin administered to said patient is 200 mg/m² or more, 250 mg/m² or more, preferably 400 mg/m² or more; when the platinum-based drug is oxaliplatin the dosage of oxaliplatin administered to said patient is 40 mg/m² or more, 60 mg/m² or more, preferably 85 mg/m² or more. In an embodiment of the invention, when the platinum-based drug is cisplatin the dosage of cisplatin administered to said patient is 20 mg/m² or more, preferably 25, 30, 35, 40, 45, 50, 55, 60, 65, 70, 75, 80, 85, 90, 95, 100, 110, 120, 130, 140, 150, 160, 170, 180, 190 or 200 mg/m² or more. In an embodiment of the invention, when the platinum-based drug is carboplatin the dosage of carboplatin administered to said patient is 200 mg/m² or more, preferably 220, 240, 250, 260, 280, 300, 325, 350, 375, 400, 450, 500, 550, 600, 650, 700, 750, 800, 850, 900, 950, 1000, 1050, 1100, 1150, 1200, 1250, 1300, 1350, 1400, 1450 or 1500 mg/m² or more. In an embodiment of the invention, when the platinum-based drug is oxaliplatin the dosage of oxaliplatin administered to said patient is 40 mg/m² or more, preferably 45, 50, 55, 60, 65, 70, 75, 80, 85, 90, 95 or 100 mg/m² or more.

In an embodiment of the invention, STS is administered between 7 hours, 6 hours, 5 hours, 4 hours, 3 hours, 2 hours, 1 hour or 0.5 hour before and 7 hours, 6 hours, 5 hours, 4 hours, 3 hours, 2 hours, 1 hour or 0.5 hour after the platinum-based drug is administered to said human subject. In a preferred embodiment the STS is administered between 3 hours prior and 3 hours after the platinum-based drug is administered to said human subject. In a particularly preferred embodiment the STS is administered between 4 hours and 0.5 hours, even more preferably between 4 and 2 hours, prior to the administration of the platinum-based drug. It is further envisioned that the STS may be provided within this period using multiple successive administrations, to further increase the total volume of administered STS composition to each ear. It is further envisioned that a first dosage of STS is provided 8 hours, preferably, 7, 6, 5, 4, 3, 2, 1, 0.5 hours before administering the platinum-based drug or 8 hours, preferably 7, 6, 5, 4, 3, 2, 1, 0.5 hours after administering the platinum-based drug, and a second or further dosages of STS are provided outside this time frame, for example 36 hours or less after the platinum-based drug is administered to said human subject or earlier, preferably 30, 24, 20, 18, 17, 16, 15, 14, 13, 12 hours or less after the platinum-based drug is administered to said human subject.

In an embodiment of the invention STS is administered to one or, preferably, to both middle ears. Preferably STS is administered to each ear of the patient separately, wherein each ear individually receives the preferred dose of STS within the preferred time window according to the invention. Preferably the STS is administered in the form of a gel to each ear.

In an embodiment of the invention the STS is comprised in a composition, preferably a gel composition, wherein the intrinsic viscosity of the composition is between 80% and 120% of the intrinsic viscosity of a composition consisting of an aqueous solution of 0.5% sodium hyaluronate and 0.1 M STS in phosphate buffered saline. It was found that a 0.5% sodium hyaluronate solution, as described herein, preferably in PBS as described herein has a suitable intrinsic viscosity for use according to the invention, however the invention should not be limited to sodium hyaluronate as the skilled person is aware that other suitable gelling agents may be used for middle ear administration. Therefore, in a preferred embodiment the intrinsic viscosity of the gel is between 80% and 120% (e.g. 80%, 85%, 90%, 95%, 100%, 105%, 110%, 115% or 120%) of the intrinsic viscosity of a gel made of 0.5% sodium hyaluronate and 0.1 M STS in phosphate buffered saline (PBS) as described herein. Preferably the gel is 37 degrees Celsius.

As provided herein, the liquid or gel composition comprising sodium thiosulfate preferably has an intrinsic viscosity between 100 m³/kg and 500 m³/kg as determined at 37 degrees Celsius. More preferably the liquid or gel composition comprising sodium thiosulfate has an intrinsic viscosity between 200 m³/kg and 420 m³/kg, most preferably between 275 m³/kg and 325 m³/kg. Alternatively, the viscosity may be determined at 25 degrees Celsius, in such case the liquid or gel composition comprising sodium thiosulfate preferably has an intrinsic viscosity between 100 m³/kg and 500 m³/kg. More preferably the liquid or gel composition comprising sodium thiosulfate has an intrinsic viscosity between 180 m³/kg and 400 m³/kg, most preferably between 250 m³/kg and 300 m³/kg. The intrinsic viscosity, and a suitable method for obtaining the intrinsic viscosity, for a pharmaceutically acceptable composition comprising STS according to the invention is provided in Example 2.

The skilled person is familiar with methods to determine the intrinsic viscosity of a liquid or a gel. A test method that can be used for determining the intrinsic viscosity of a composition, for example of the composition according to the invention, as disclosed herein is a method for the Intrinsic Viscosity by Capillary flow according to Ph.Eur. 2.2.8/2.2.9 and further in the applicable sodium hyaluronate monograph (January 2017:1472). In said method the flow times of 4 dilutions of test material are determined in a suitable capillary in an appropriate suspended level viscometer. As provided herein the intrinsic viscosity is calculated by linear least squares regression analysis of flow times against the concentration of the samples using the Martin equation. The skilled person is well aware of other suitable test methods. The skilled person is familiar with similar methods of calculating intrinsic viscosity.

Further, as disclosed herein, viscosity is measured by equipment such as a Capillary Viscometer, for example a Capillary Viscometer Ubbelohde, or similar equipment known to a skilled person.

In an embodiment of the invention the STS is administered at a temperature between 15 degrees Celsius and 40 degrees Celsius, preferably between 20 degrees Celsius and 37 degrees Celsius or preferably between 35 degrees Celsius and 38 degrees Celsius. Preferably the temperature is close to the body temperature of patient. A too low or too high temperature is likely to cause caloric events as dizziness and vertigo to the patient and may make the gel too viscous to be able to inject it. A too high temperature may also result in burning of the tissue.

In an embodiment of the invention the STS is comprised in a composition, preferably gel composition, and wherein STS is administered to the middle ear in a volume of at least 0.05 ml, with increasing preference at least 0.1 ml, 0.2 ml, or 0.3 ml and/or with a volume of at most 5.0 ml, with increasing preference at most 4.0 ml, or 3.0 ml.

Without wishing to be bound by theory, it is theorized that a higher injection volume results in better filling of the middle ear with the STS, resulting in better local uptake of the STS. Therefore, the volume of the STS comprising composition which is administered to each ear individually is preferably at least 0.05 ml, for example at least 0.06 ml, 0.07 ml, 0.08 ml, 0.09 ml, 0.1, 0.12 ml, 0.14 ml, 0.16 ml, 0.18 ml, 0.2 ml, 0.22 ml, 0.24 ml, 0.26 ml, 0.28 ml, 0.3 ml, 0.32, 0.35 ml, 0.38 ml, 0.4 ml, 0.42 ml, 0.45 ml, 0.5 ml, 0.6 ml, 0.7 ml, 0.8 ml 0.9 ml, 1.0 ml, 1.1 ml, 1.2 ml, 1.3 ml 1.4 ml, 1.5 ml, 1.6 ml, 1.7 ml, 1.8 ml, 1.9 ml, 2.0 ml, 2.1 ml, 2.2 ml, 2.3 ml 2.4 ml, 2.5 ml, 2.6 ml, 2.7 ml, 2.8 ml, 2.9 ml, 3.0 ml, 3.2 ml, 3.4 ml, 3.6 ml, 3.8 ml, 4.0 ml, 4.2 ml, 4.4 ml, 4.6 ml or at least 4.8 ml. Preferably the volume does not exceed the volume of the cavity of the middle ear, therefore preferably the volume is at most 5.0 ml, 4.8 ml, 4.6 ml, 4.4 ml, 4.2 ml, 4.0 ml, 3.8 ml, 3.6 ml, 3.4 ml, 3.2 ml or at most 3.0 ml. The skilled person understands that also multiple small administrations with STS may be provided to the patient.

In the Rolland study no significant results were obtained upon STS administration. In that study a small volume of gel, 0.1 ml, was administered having a high concentration of STS (0.5 M) 24 hours prior to cisplatin was provided. It has now surprisingly been found that using a larger volume, even if the absolute amount of STS remains similar, results in much improved reduction or prevention of hearing loss. Therefore, in a preferred embodiment the volume which is injected in the middle ear is preferably more than 0.1 ml, preferably more than 0.2 ml more preferably more than 0.3 ml. The administration of larger volumes of gel potentially carries a higher potential risk of adverse or undesired effect, for example in the form of caloric events due to increased thermal mass within the middle ear that potentially evokes said caloric response. For example, a large volume of gel combined with a too low temperature of the gel is potentially more likely to cause caloric events as dizziness and vertigo to the patient and a large volume of gel combined with a too high temperature of the gel is potentially more likely to result in heat damage of tissue. Therefore, in a preferred embodiment the volume which is injected in the middle ear of preferably more than 0.1 ml, preferably more than 0.2 ml more preferably more than 0.3 ml, has a temperature that is close to the body temperature of the human subject, preferably the temperature of the administered solution is between 15 degrees Celsius and 40 degrees Celsius, more preferably between 20 degrees Celsius and 37 degrees Celsius or between 35 degrees Celsius and 38 degrees Celsius.

In an embodiment of the invention the middle ear is vented when STS is administered and/or wherein STS is administered to the middle ear while venting the middle ear, preferably wherein venting is via an opening, preferably a grommet, present in the eardrum, and preferably wherein STS is administered to the middle ear not through said opening. It was found that venting the middle ear allowed for the injection of a greater volume into the middle ear. It may be desirable to inject a greater volume, as it was found by the inventors that a greater volume contributes to better protection against platinum-based drug induced hearing loss, possibly due to the better filling of the middle ear when a greater volume is used.

In an embodiment of the invention the STS is comprised in a pharmaceutical acceptable composition, preferably a gel composition, in a concentration of between 0.025 M and 1.0 M, preferably between 0.05 M and 0.5 M, 0.05 M and 0.2 M, more preferably between 0.075 M and 0.15 M. The skilled person will appreciate that the total dose to be administered is a function of the volume to be administered and the concentration. Therefore, if a larger volume is to be administered, the concentration may be reduced so the same amount of STS is administered and vice versa.

In a particularly preferred embodiment the invention relates to sodium thiosulfate (STS) for use in preventing or reducing hearing loss induced by a platinum-based drug wherein STS is administered to the middle ear of a human subject between 4 to 2 hours before the platinum-based drug is administered to said human subject, wherein said STS is comprised in a pharmaceutically acceptable gel, wherein the intrinsic viscosity of said gel is between 80% and 120% of the intrinsic viscosity of a composition consisting of an aqueous solution of 0.5% sodium hyaluronate, wherein said gel comprises between 0.05 M and 0.2 M STS, and wherein said STS is administered to the middle ear in a volume of at least 0.3 ml.

In an embodiment of the invention there is provided a platinum-based drug for use in preventing or reducing hearing loss induced by the platinum-based drug and/or for use in the treatment of cancer, wherein STS is administered to the middle ear of a human subject between 8 hours before and 8 hours after the platinum-based drug is administered to said human subject.

Further provided herein is an improved method of preventing or reducing hearing loss induced by the platinum-based drug or treatment of cancer, the method involving administering STS prior or after administering a platinum-based drug. The benefit of the claimed method being reduced side-effects of said platinum-based drug administration, as the administration of STS prior or after the administration of the platinum-based drug as claimed reduces hearing-loss. Preferably the STS is administered as described herein, preferably as a pharmaceutically acceptable gel composition, preferably a gel composition as described herein.

In an embodiment of the invention is provided a composition, preferably a gel composition, comprising STS and/or a composition comprising a platinum-based drug for use in preventing or reducing hearing loss induced by the platinum-based drug wherein STS is administered to the middle ear of a human subject between 8 hours before and 8 hours after the platinum-based drug is administered to said human subject. Preferably the STS is administered as described herein, preferably as a pharmaceutically acceptable gel composition, preferably a gel composition as described herein.

In an embodiment of the invention is provided a kit of parts comprising:
a container, preferably an ampoule, a syringe and/or an injection needle, comprising a pharmaceutical acceptable composition, preferably a gel composition, comprising STS, wherein said container, preferably an ampoule, a syringe and/or an injection needle is for administration of between 0.05 and 5.0 ml of said composition to the middle ear of a human subject, preferably wherein the said container, preferably an ampoule, a syringe and/or an injection needle is for single-use; and/or
a first container, preferably an ampoule, comprising an aqueous solution comprising STS and a second container, preferably a syringe and/or an injection needle, comprising a gel (pre-gel), preferably a gel with a composition consisting of: an aqueous solution of sodium hyaluronate.

The invention further provides for a kit of parts, suitable for administering STS to a patient. The STS may be provided as ready to use gel, or the gel and the STS may be provided in separate containers and need to be mixed prior to administration.

In case the STS is provided as a ready to use gel it is preferably already present in the concentration to be administered, for example between 0.05 M and 0.2 M, preferably between 0.075 M and 0.15 M most preferably around 0.1 M. More preferably the STS is present in a gel which has an intrinsic viscosity which is between 80% and 120% of the intrinsic viscosity of a gel composition consisting of 0.5% sodium hyaluronate (e.g. in PBS as described herein). In a particularly preferred embodiment, the gel is an aqueous solution of between 0.25% and 1.0% sodium hyaluronate, preferably between 0.4% and 0.6%. Preferably the STS gel composition is in an ampoule, more preferably in a syringe, preferably a syringe suitable for transtympanic or intratympanic administration of said gel composition into the middle ear of a human subject. Preferably the gel composition further comprises phosphate buffered saline (PBS).

Alternatively, the kit of parts may contain separate containers, one comprising an STS composition and one comprising a pre-gel, which need to be combined prior to administration. The skilled person will appreciate that the concentration of STS and the viscosity of the pre-gel need to be higher, so that when combined they have the desired concentration and viscosity. For example, if it is desired that the final STS concentration is 0.1 M and the pre-gel and the STS composition are to be mixed in a 1:1 ratio, the STS composition prior to mixing with the pre-gel should be 0.2 M.

The following preferred embodiments assume that the STS composition and the pre-gel are mixed in a 1:1 ratio prior to administration, it is well within the skilled person's ability to change these numbers in case a different ratio of mixing is preferred, for example 5:1, 4:1, 3:1, 2:1, 1:2, 1:3, 1:4, or 1:5. Preferably the STS composition prior to mixing with the pre-gel has a concentration of between 0.1 M and 0.4 M, preferably between 0.15 M and 0.3 M most preferably around 0.2 M. More preferably the pre-gel, after mixing with the STS composition, provides an intrinsic viscosity which is between 80% and 120% of the viscosity of a gel composition consisting of 0.5% (w/w) sodium hyaluronate, e.g. as described herein. In a particularly preferred embodiment, the pre-gel is an aqueous gel of between 0.5% and 2.0% sodium hyaluronate, preferably between 0.8% and 1.2%. Preferably the STS composition, and/or the pre-gel is in an ampoule, more preferably the pre-gel in a syringe and the STS composition is in an ampoule or vice versa, preferably said syringe is suitable for transtympanic or intratympanic administration of said gel composition into the middle ear of a human subject. Preferably the STS composition further comprises phosphate buffered saline (PBS) and has a pH of 7.4.

In an embodiment of the invention is provided a method of preventing or reducing hearing loss induced by a platinum-based drug in a human subject, the method comprising that STS is administered to the middle ear of a human subject between 8 hours before and 8 hours after the platinum-based drug is administered to said human subject. Preferably the STS is administered as described herein, preferably as a pharmaceutically acceptable gel composition, preferably a gel composition as described herein.

In an embodiment of the invention the human subject has cancer.

### EXAMPLES

### Example 1—Intratympanic Application of STS

Methods

Study Design

This phase 1 trial was performed in two STS treatment arms. Arm A was a single-blind, placebo controlled, proof-of-concept study. A different application method was investigated in arm B, which was a non-blinded, non-placebo controlled, proof of concept arm. The ear to be treated with the transtympanic STS gel was assigned by randomisation in both arms. The study was performed at the Netherlands Cancer Institute in Amsterdam, the Netherlands. The protocol was approved by the institutional medical research ethics committee. The study is registered with the European Clinical Trials Database (EudraCT) (EudraCT: 2012-004653-80).

Patients

Patients of 18 years or older who were to be treated with cisplatin at a dose of 75 mg/m² for a solid tumour were eligible. If patients were to receive concomitant radiotherapy for HNSCC, the total radiotherapy dose on the cochlea and/or the middle ear was not allowed to exceed 30 Gray (Gy) to avoid radiotherapy-induced hearing loss. Exclusion criteria were: known symptomatic brain or leptomeningeal metastases and relevant otological history (e.g. conductive hearing loss, vertigo). All patients gave written informed consent prior to start of study procedures.

Randomisation

The ear to be treated with the transtympanic STS gel was assigned by randomisation in both arm A and arm B. Arm A concerned a single-blind study protocol in which the contralateral ear was treated with a placebo gel. Arm B was non-blinded and the contralateral ear was left untreated. The enrolment of patients was done by a treating physician. Randomisation was done via a randomisation program at the institutional trial centre.

Procedures

In arm A, on the day of cisplatin administration, two syringes with 2.0 mL 0.5% sodium hyaluronate (HYA) based gels were prepared by the institutional pharmacy: one without STS (the placebo) and one with 0.1 M STS. In arm B only the STS gel was prepared. Syringes with study medication were warmed up to body temperature (37° C.) for 30 minutes in an incubator (CULTURA® M, Almedica AG, Giffers, Switzerland) to prevent hypo- or hypercaloric symptoms during or after injections. The syringe was connected to the (Braun, Pencan 25G) needle via a 10 cm infusion line (BD Becton Dickenson connecta). The needle and tip of the injection needle were bended to 1. Approach the ear drum under sight, and 2. To approach the eardrum in a perpendicular orientation.

In arm A, the gels were administered through a preplaced grommet, which was placed for venting air to prevent barotrauma and/or pain while injecting. During this procedure we noticed there was backflow of the gel via the infusion needle backward into the external ear canal. Therefore, a different protocol for the transtympanic injection was chosen in arm B for the next 6 patients: the grommet was still placed in the anterior eardrum for ventilation of the middle ear, however, the STS gel was injected via the needle directly through the posterior part of the eardrum and indeed backflow into the external ear was not seen anymore.

Topical anesthesia was applied using a few small (3×3 mm) gauzes with xylocaine 10% (lidocaine 100 mg/ml, AstraZeneca, United Kingdom) applied on the eardrum before placement of the grommet and the injections. During the injections, the patient was lying in a hospital bed with the upper body positioned 30 degrees upwards. After administration of the gel the patients head was turned 45 degrees contralateral. Patients remained in this position for 30 minutes and were instructed to keep swallowing and talking to a minimum. The specific positioning of the patient was in our view needed to for improved and/or prolonged exposure of the STS gel to the round window of the middle ear.

Cisplatin infusion was started three hours after STS administration. If concomitant chemotherapy or radiotherapy was to be administered, this was done as per local protocol. Follow-up was performed within 7 days prior to start of each cisplatin cycle, and within 1 and 3 months after the last cycle and consisted of audiometry, physical examination, registration of adverse events and safety laboratory assessments consisting of hematology and serum chemistry.

Outcomes

The primary outcome of this study was the safety and feasibility of intratympanic application of STS gel. Secondary outcomes were the efficacy of this modality against CIHL and the pharmacokinetics of cisplatin in plasma. Adverse events were registered according to the Common Terminology Criteria for Adverse Events version 4.03 (CTCAE).

Standard audiometry including air conduction (AC) and bone conduction (BC) thresholds was performed in a sound proof booth using the Decos Audiology Workstation. We calculated three Pure Tone Averages (PTAs) at three different frequency ranges: for one range relevant for speech perception in quiet we took the average threshold at 0.5-1-2 kHz (further referred to as PTA1), for speech perception in noise at 1-2-4 kHz (PTA2) and for the perception of high sounds at 8-10-12.5 kHz (PTA3). In some patients, the thresholds at 8 kHz were not available for ultrahigh frequency audiometry and we converted these values from the regular pure-tone audiometry thresholds into dB SPL following ISO 389-1. If the hearing level was untestable as it was beyond the audiometer's maximum output, we computed the threshold by adding 5 dB to this maximum (i.e. 100+5=105 dB or 110+5=115 dB, depending on the settings of the audiometer). Audiometric testing was performed pre-treatment (referred to as baseline), after each cisplatin cycle and 3 months posttreatment (follow-up). To correct for fluctuating conductive components, we took the BC thresholds for 0.5, 1, 2 and 4 kHz if the averaged threshold at these frequencies together at AC differed ≥10 dB from the averaged threshold at these frequencies at BC.

To evaluate efficacy, clinically relevant treatment-related SNHL was defined as 10 dB SNHL starting at PTA3. Hearing loss of <10 dB is generally accepted to be clinically insignificant, according to the CTCAE version 4.03 and ASHA guidelines. In addition, a clinically relevant response to STS was defined as ≥10 dB difference starting at PTA3 after treatment in favor of the STS-treated ear when compared to the not STS-treated ear. Next, patients were divided into three groups: 1) patients that unexpectedly did not develop any cisplatin-related SNHL, 2) patients that developed SNHL and responded to the STS gel, and 3) patients that developed SNHL but did not respond to the STS gel.

PK sampling was performed. Samples were drawn pre-dose, at end of infusion and 1, 2, 3, 4, and 18 hours thereafter. Blood was collected in a 10.0 mL heparin tube. After collection, the tube was centrifuged at 1500 g for 10 minutes at 4° C. From the plasma 2.0 mL was transferred to a 2.0 mL Eppendorf tube and stored at −20° C. (total platinum). A plasma ultra-filtrate tube (Centrifree® ultra-filtrate tubes, Merck Millipore Ltd, Tullagreen, Ireland) was filled with plasma and centrifuged at 1800 g for 10 minutes. The ultra-filtrate was then transferred to a 2.0 mL Eppendorf and stored at −20° C. (unbound platinum). Platinum levels were measured by the pharmacy of the Netherlands Cancer Institute using two validated inductively coupled plasma mass spectrometer methods (ICP-MS). The lower level of quantification was 7.50 ng/L. PK parameters were calculated using validated scripts in the software package R version 3.0.1. The maximum observed plasma concentration (Cₘₐₓ), the area under the plasma concentration time curve from start of the cisplatin infusion (time=0) to 22 hours (AUC_{0-22 h}) were reported.

Statistical Analysis

Six evaluable patients per study arm were considered sufficient to reach proof of concept based on the intra-patient comparison and the expected audiometric difference between the STS-treated and untreated/placebo treated ears. The low standard errors for audiometric differences before and after cisplatin that were shown in a previous study allowed us to use a small number of patients. To study whether there was a significant difference in CIHL over time (ΔPTA3) between the STS ears and the untreated ears, the Mann Whitney U test with paired samples was used. The difference in the incidence of ototoxicity (defined as ΔPTA3≥10 dB) between the STS ears and untreated ears was analyzed by the McNemar test. P-values of ≤0.05 were considered statistically significant.

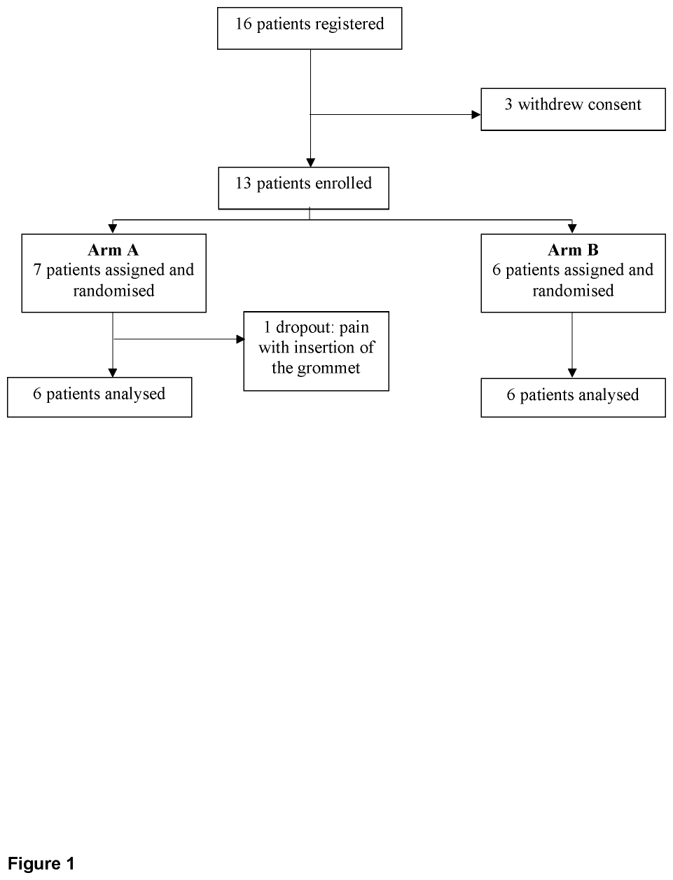

In total, 16 patients were registered (FIG. 1), however 4 of patients withdrew consent and were excluded for the below reasons: Three patients did not start therapy: one experienced pain from bone metastases, one considered logistics to be troublesome and one without a formal reason. The fourth patient with a narrow ear found insertion of the grommets painful and withdrew consent after inclusion but before treatment in ARM A of the trial. Patients were treated between June 2013 and October 2018. For baseline patient characteristics see table 1. Eight patients were male (67%). Patients had a median age of 60 (range 46-67) years. In two patients, cisplatin was discontinued after one cycle due to nephrotoxicity: in one of them cisplatin was switched to carboplatin and the other patient with HNSCC was switched from radiotherapy with concomitant cisplatin to radiotherapy alone.

In total, 34 STS injections were given. In arm A, an averaged gel volume of 0.2 mL (range, 0.1-0.3 mL) was injected. In this treatment arm, application of the gel via the needle inserted in the middle ear through the grommet was unfortunately limited by backflow of the gel backward in the external ear canal alongside that very same needle. The technique was improved in arm B, where the gel was injected by direct transtympanic puncture of the posterior eardrum: a larger volume with a mean of 0.37 mL (range, 0.3-0.5 mL) STS gel could be administered. In arm B, a clear increasing “water level” could be observed behind the transparent eardrum while injecting.

Directly after insertion of the grommet and application of the gel(s) temporary and modest side-effects were reported. Almost all patients reported upon request that they experienced that their perception of sound had slightly changed in quality (not quantity) after placement of the grommet, which could not be objectified by audiometry and could therefore not be scored according to CTCAE version 4.03. One patient needed to receive subcutaneous local anesthesia with lidocaine 2% before insertion of the grommet and experienced vertigo directly afterwards, which diminished within four hours. Generally, transtympanic injections were well tolerated. One patient with a narrow ear canal reported that the administration of the gel was “a bit painful”, but continued therapy. Some patients reported fullness of the middle ear directly after application of the gel, which resolved spontaneously within 1 hour after injection. No persistent otitis media, perceptive hearing loss or vertigo as a result of gel application occurred in the study. No adverse event (AE) occurred that were considered (possibly, probably or definitely) related to the gel application. All reported serious adverse events (SAEs) were to be attributed to cisplatin doublet treatment or to malignant disease and unrelated to STS injection. These six SAEs (CTCAE grade 2 and 3) occurred in four patients with HNSCC treated with concomitant radiotherapy and 100 mg/m² cisplatin and included hospitalization due to increased serum creatinine levels, neutropenia or dehydration.

The quantity of audiometric data that have been converted from the threshold at 8 kHz measured with regular pure-tone audiometry into dB SPL was equal in both groups. Also, the number of thresholds that have been computed because they exceeded the audiometer's maximum output was nearly even in both groups.

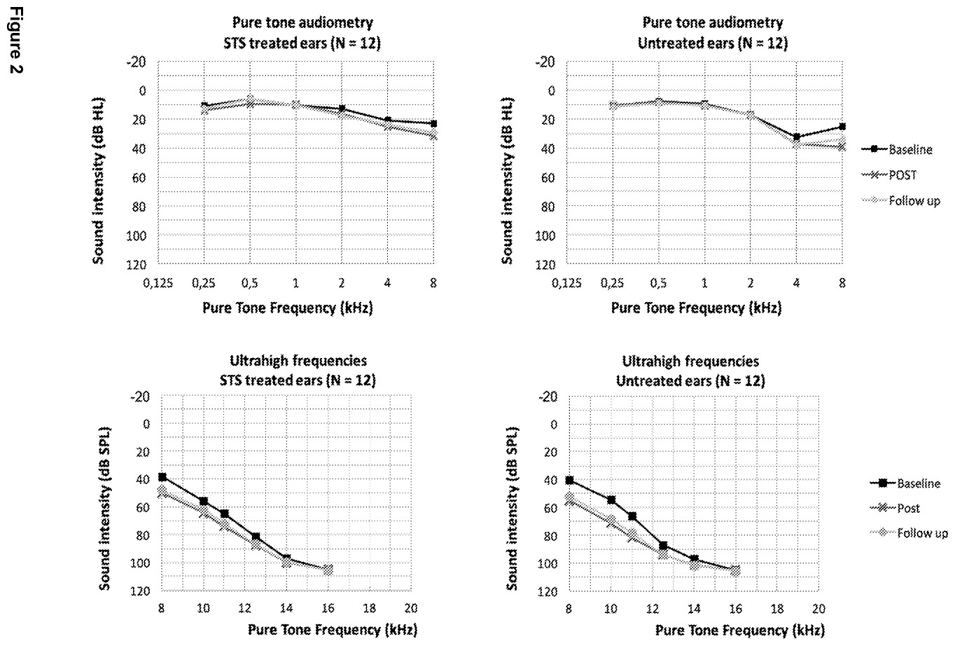

For all 12 patients, the mean AC thresholds for the STS treated ears and the untreated/placebo ears as measured at baseline, directly post cisplatin treatment and at follow-up are depicted in FIG. 2. Overall, ΔPTA3 was 12.7 dB in the untreated ears and 8.8 dB in the STS-treated ears (p=0.413, Mann Whitney U). Overall, there was no statistically significant difference in the occurrence of SNHL (defined as ΔPTA3 of ≥10 dB in one or both ears) between the 12 STS-treated ears and 12 untreated ears (p=0.125, McNemar).

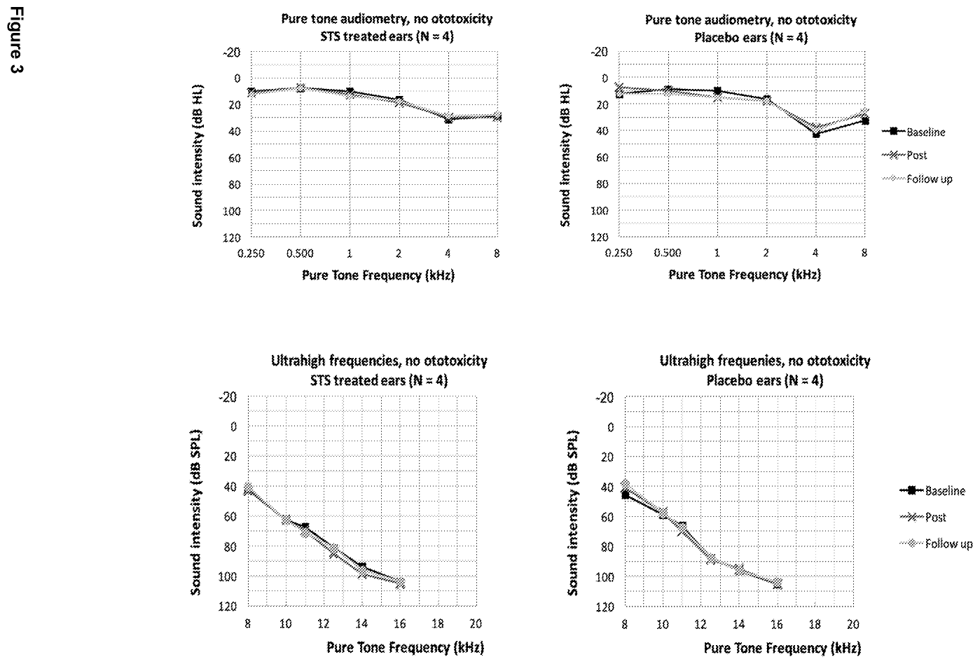

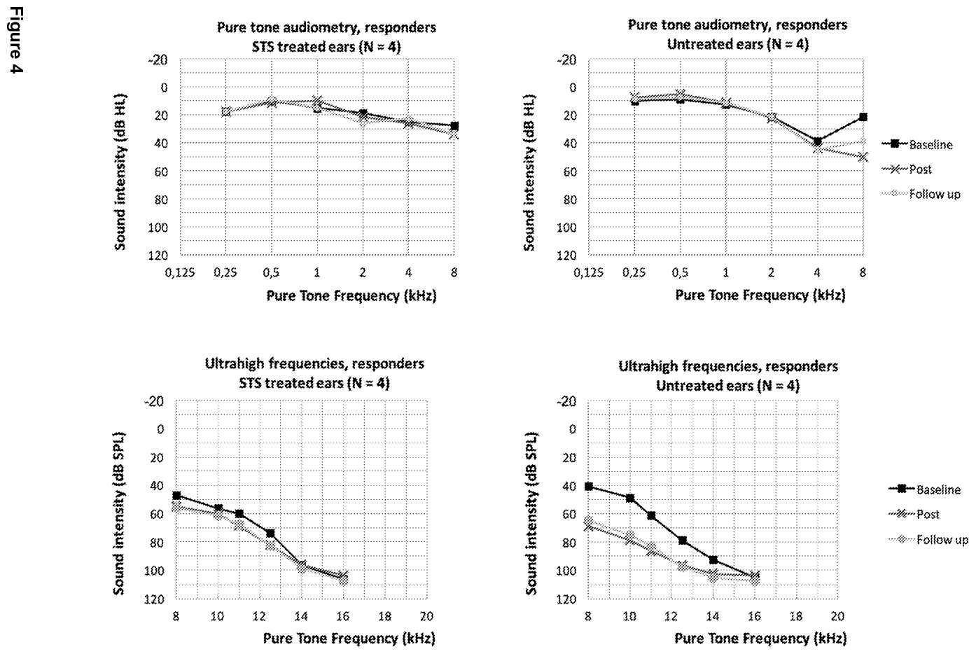

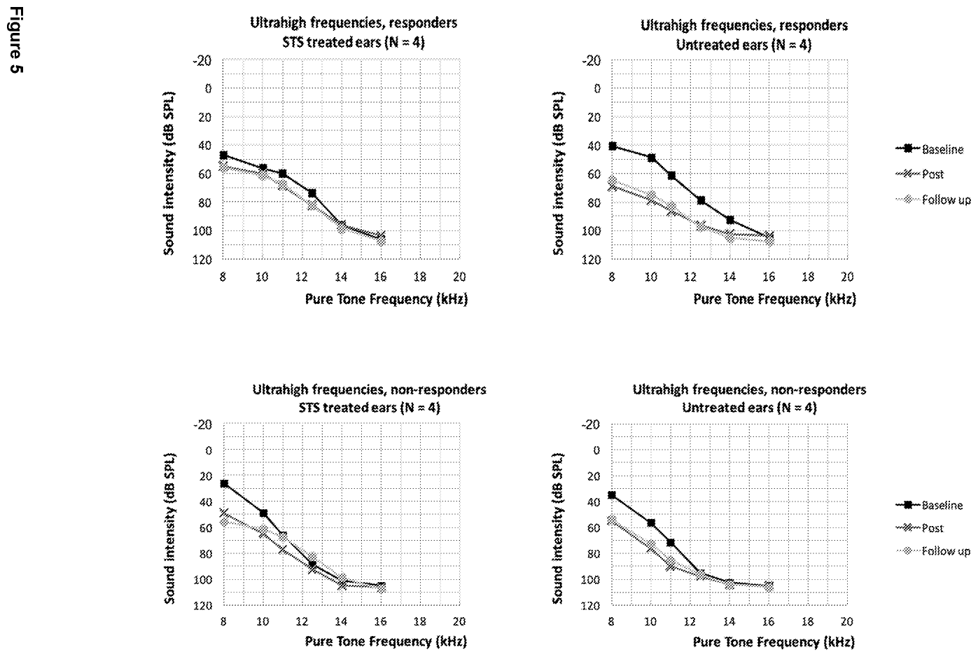

Patients were divided into three groups: 1) patients that unexpectedly did not develop SNHL, 2) patients that developed CIHL and responded to the STS gel, and 3) patients that developed SNHL but did not respond to the STS gel. In total, 4 patients (all treated in arm A, treated with cisplatin 75 mg/m² in 1-4 cycles) did unexpectedly not develop CIHL, see FIG. 3. Eight patients did develop CIHL, of whom four (50%) responded to the STS gel (FIG. 4, Table 3) and four (50%) did not respond to the STS gel (FIG. 5). In the four responders, there was a difference in ΔPTA3 between the STS-treated ears and untreated ears of 18.4 dB (range, 11.7-33.3 dB), as the mean ΔPTA3 was 6.8 dB in STS-treated ears and 25.2 dB in the untreated ears (p=0.125, Mann Whitney U). Prior to confirmation by audiometric evaluation, one of the STS-responding subjects, who was a sound engineer by profession, reported that not only hearing capacity was better in his STS-treated ear, but also the quality of sound. In the four non-responders, ΔPTA3 was 14.3 dB in the STS-treated ears and 15.2 dB in the untreated ears. In three of them, ΔPTA3 differed <10 dB SPL between ears. In the last non-responder, directly after treatment, the difference in ΔPTA3 was 10.0 dB SPL in favour of the untreated ear, but this diminished to 2.3 dB SPL at follow-up audiometry. The grouped non-responders includes one patient treated in arm A who received an estimated volume of 0.1 mL STS gel during all 3 injections due to backflow of the gel through the grommet into the ear canal. Another patient in the group of non-responders might have been unable to rest for 30 minutes in the desired position, as he experienced nausea after receiving anaesthetics with lidocaine 2% injection and subsequent movements might have caused difficulties with the exposure or absorption of the gel by the round window.

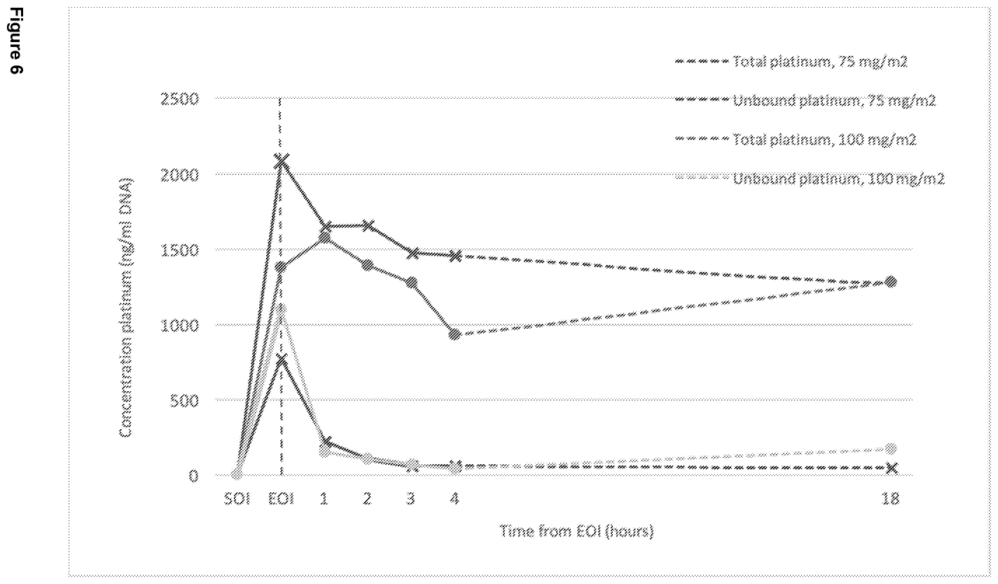

Concentration-time curves for total and unbound platinum are presented in FIG. 6. Corresponding PK parameters Cₘₐₓ and AUC_{0-18 h} are shown in table 4. The PK data of one patient treated with 100 mg/m² are missing, as PK samples were not taken due to logistic reasons.

A cisplatin dose of >75 mg/m² was administered. When considering an average body surface area of 1.8 m², patients received about 0.43 mmol cisplatin. The amount of STS administered ranged from 0.01-0.05 mmol. Because STS binds to platinum in a 1:1 ration, less than 10% of the molar weight of the administered cisplatin dose could be neutralized. Nevertheless, because STS has a poor oral bio-availability, only a minority of the injected STS will be available in the systemic circulation.27 A comparison of our PK results with previous literature is difficult, as it was previously shown that STS-bound platinum may be detected as unbound platinum by the ICP-MS method.28 A comparison of the unbound fraction is therefore not useful. However, the levels observed were in line with results of a previous report. Based on literature and our PK data, we postulate that transtympanic STS does not interfere with the systemically available cisplatin.

The application of transtympanic STS gel was safe and feasible in adults treated with cisplatin in this phase I/II randomized clinical trial. Generally, CIHL is characterized by symmetric and irreversible SNHL.1,24 In our phase I trial, four out of eight patients enduring clinically relevant CIHL showed hearing capacities in SPL at PTA 8-10-12.5 kHz after cisplatin chemotherapy in favor of their STS-treated ear of 18.4 dB (range, 11.7-33.3 dB) difference when compared to their untreated ear. Therefore, although not statistically significant, the results of this trial indicate that we have observed—in single patients—the first in human and clinically relevant efficacy of transtympanic application of STS.

### TABLES

TABLE 1
Baseline characteristics.

| | Arm A | Arm B | Total |
|---|---|---|---|
| | (n = 6) | (n = 6) | (n = 12) |
| Age | | | |
| Median (range) | 60 (46-67) | 59 (46-63) | 59 (36-67) |
| | years | years | years |
| Sex | | | |
| Male | 5 (83%) | 3 (50%) | 8 (67%) |
| Female | 1 (17%) | 3 (50%) | 4 (33%) |
| WHO Performance | | | |
| Score | | | |
| 0 | 5 (83%) | 6 (100%) | 11 (92%) |
| 1 | 1 (17%) | 0 | 1 (8%) |
| Tumour type | | | |
| NSCLC | 3 (50%) | 1 (17%) | 4 (33%) |
| Mesothelioma | 3 (50%) | 0 | 3 (24%) |
| Thymus carcinoma | 0 | 1 (17%) | 1 (8%) |
| HNSSC | 0 | 4 (67%) | 4 (33%) |
| Number of cycles | | | |
| cisplatin | | | |
| Median (range) | 3 (2-4) | 3 (1-4) | 3 (1-4) |
| | cycles | cycles | cycles |
| Cisplatin dose | | | |
| 75 mg/m² | 6 (100%) | 2 (33%) | 8 (67%) |
| Number of cycles, | 3 (1-4) | 4 (4) | 3.5 (1-4) |
| median (range) | cycles | cycles | cycles |
| 100 mg/m² | 0 | 4 (67%) | 4 (33%) |
| Number of cycles, | 0 | 2 (1-3) | 2 (1-3) |
| median (range) | | cycles | cycles |

Abbreviations: HNSSC = head and neck squamous cell carcinoma; NSCLC = non-small cell lung cancer; WHO = World Health Organization.

TABLE 2
Differences between STS-treated ears and untreated ears in
pure tone averages (PTAs) over time (Δ) of all 12 patients.

| | Δ (posttreatment − baseline) | | |
|---|---|---|---|
| PTA (kHz) | | STS-treated ears | Untreated ears |

| | | | | |
|---|---|---|---|---|
| 0.5-1-2 | 2.1 | dB HL | 0.7 | dB HL |
| 1-2-4 | 2.5 | dB HL | 2.2 | dB HL |
| 8-10-12.5 | 8.8 | dB SPL | 12.7 | dB SPL |

The differences in Δ PTA in dB (Δ PTA = PTA directly after the last cycle of cisplatin minus baseline PTA).
Abbreviations: HL = hearing level; kHz = kilohertz; PTA = pure tone average; SPL = sound pressure level; STS = sodium thiosulfate.

TABLE 3
Differences between STS-treated ears and untreated ears
in pure tone averages (PTAs) over time (Δ) of the
4 patients that did developed ototoxicity (defined as
a Δ PTA 8-10-12.5 ≥10 dB) and responded to the STS-gel.

| | Δ (posttreatment − baseline) | | |
|---|---|---|---|
| PTA (kHz) | | STS-treated ears | Untreated ears |

| | | | | |
|---|---|---|---|---|
| 0.5-1-2 | −0.4 | dB HL | −1.3 | dB HL |
| 1-2-4 | −0.4 | dB HL | 1.7 | dB HL |
| 8-10-12.5 | 6.8 | dB SPL | 25.2 | dB SPL |

Response is defined as patients with ototoxicity in which Δ PTA 8-10-12.5 kHz in the untreated ear exceeds Δ PTA 8-10-12.5 kHz in the STS-ear with ≥10 dB (Δ PTA = PTA directly post-treatment minus baseline PTA).
Abbreviations: HL = hearing level; kHz = kilohertz; PTA = pure tone average; SPL = sound pressure level; STS = sodium thiosulfate.

TABLE 4
Pharmacokinetic parameters.

| | | Cisplatin 75 mg/m² (n = 8) | | Cisplatin 100 mg/m² (n = 3) | |
|---|---|---|---|---|---|
| | | Total | Unbound | Total | Unbound |
| | | platinum | platinum | platinum | platinum |
| Cₘₐₓ | Mean ± SD | 2085.8 ± 566.3 | 771.5 ± 176.2 | 1906.7 ± 306.2 | 1098 ± 51.8 |
| ng/ml | (CV) | (27.1%) | (22.8%) | (16.1%) | (4.7%) |
| AUC_{0-22 hrs} | Mean ± SD | 29770.0 ± 5879.3 | 3016.6 ± 586.7 | 22386.6 ± 6636.8 | 4288.6 ± 1852.3 |
| ng/ml*h | (CV) | (19.7%) | (19.5%) | (28.4%) | (43.2%) |

Abbreviations: Cmax = maximum observed concentration, AUC_{0-22 hrs} = area under the plasma concentration time curve from t = 0 to 22 hours, n = number of patients, SD = standard deviation, CV = coefficient of variatiation.

### Example 2—Determining Intrinsic Viscosity

Methods & Equipment

For determining the intrinsic viscosity three STS formulations and a dilution buffer were provided. The following formulations were provided:
0.1M sodium thiosulfate (STS) in 0.5% hyaluronic acid gel
0.1M sodium thiosulfate (STS) in 0.75% hyaluronic acid gel
0.1M sodium thiosulfate (STS) in 0.25% hyaluronic acid gel

The following dilution buffer was provided:
0.1M sodium thiosulfate in PBS

Viscosity was measured in a Capillary Viscometer Ubbelohde (k=0.005048 mm³/s²). A water bath (ALAN-268) was used for temperature conditioning. The temperature was checked using a thermometer (LAN-414). A shaking table (ALAN-083) was used for sample dilution homogenization.

Results

Initial Testing

The initially provided material was used to determine the appropriate sample dilutions for viscosity measurement as follows:

TABLE 5
Initial dilution data

| | | Flow time | | Flow time | |
|---|---|---|---|---|---|
| | | at 25° C. | | at 37° C. | |
| Test sample | Dilution | (sec) | Ratio | (sec) | Ratio |
| Dilution | N/A | 184.87 | N/A | 146.42 | N/A |
| 0.1M STS | 1 to 18.75 | 294.70 | 1.6 | 226.37 | 1.6 |
| in 0.75% | 1 to 9.375 | 338.46 | 2.3 | 439.82 | 2.4 |
| hyaluronic | | | | | |
| acid gel | | | | | |
| Criterion | | | 1.6-1.8 | | 1.6-1.8 |

The results in the Table 5 show that acceptable dilutions could be made using a dilution of 1 in 18.75 for 0.1M sodium thiosulfate in 0.75% hyaluronic acid gel. From this result the following dilutions were estimated:
0.1M STS in 0.25% hyaluronic acid gel: 6× dilution (required minimal 20 g)
0.1M STS in 0.5% hyaluronic acid gel: 10× dilution (required minimal 10 g)
0.1M STS in 0.75% hyaluronic acid gel: 10× dilution (required minimal 6 g)

Final Sample Analysis

Using these dilutions the three formulations were tested to fall within the prescribed range (ratio to buffer 1.6-1.8). The following Table 6 presents the results of the flow ratios:

TABLE 6
Dilution data

| | | Flow time | Ra- | Flow time | Ra- |
|---|---|---|---|---|---|
| | Dilution | @25° C. | tio@25° | @37° C. | tio@37° |
| Test sample | A | (sec) | C. | (sec) | C. |
| Dilution | N/A | 184.73 | N/A | 146.32 | N/A |
| 0.1M STS | 20.0 g | 327.24 | 1.77 | 252.53 | 1.73 |
| in 0.25% | sample + | | | | |
| hyaluronic | 100.0 g | | | | |
| acid gel | buffer | | | | |
| 0.1M STS | 20.0 g | 340.23 | 1.84 | 268.19 | 1.83 |
| in 0.5% | sample + | | | | |
| hyaluronic | 100.0 g | | | | |
| acid gel | buffer | | | | |
| 0.1M STS | 6.7 g | 340.51 | 1.84 | 268.13 | 1.83 |
| in 0.75% | sample + | | | | |
| hyaluronic | 100.4 g | | | | |
| acid gel | buffer | | | | |
| Criterion | | | 1.6-1.8 | | 1.6-1.8 |

Table 6 shows that for all formulations acceptable ratios were obtained at both 25 degrees Celsius and 37 degrees Celsius measurement temperatures. From the initial dilutions A, as presented in the Table, the three further dilutions were prepared according Ph. Eur. as follows:
Dilution B: 30 g Dilution A+10 ml buffer
Dilution C: 20 g Dilution A+20 ml buffer
Dilution D: 10 g Dilution A+30 ml buffer

The flow times were determined in all dilutions (A, B, C, D). The following Table 7 presents the results of the regression analysis.

TABLE 7
Regression data (Concentration (%) versus
Log (Flow time/Concentration))

| | 25° C. | | | 37° C. | | |
|---|---|---|---|---|---|---|
| | Slope | Intercept | | Slope | Intercept | |
| Test sample | (a) | (b) | R² | (a) | (b) | R² |
| 0.1M sodium | 282.1 | 2.59 | 0.952 | 248.4 | 2.61 | 0.916 |
| thiosulfate in | | | | | | |
| 0.25% hyaluronic | | | | | | |
| acid gel | | | | | | |
| 0.1M sodium | 53.0 | 2.45 | 0.999 | 40.1 | 2.48 | 0.996 |
| thiosulfate in | | | | | | |
| 0.5% hyaluronic | | | | | | |
| acid gel | | | | | | |
| 0.1M sodium | 26.8 | 2.29 | 0.983 | 21.4 | 2.31 | 0.994 |
| thiosulfate in | | | | | | |
| 0.75% hyaluronic | | | | | | |
| acid gel | | | | | | |

Table 7 shows that linear regression analysis resulted in decreasing data for Slope and Intercept with increasing concentration. The Intercept was only marginally decreased with decreasing concentration. The coefficient of determination (R²) was good for most determinations (≥0.98) but less for the 0.25% determinations (0.916-0.952).

Using the intercepts of the flow times determined by linear regression analysis the intrinsic viscosity could be calculated by taking the power-10 antilog.

The following Table 8 presents the results of the calculations:

TABLE 8
Intrinsic viscosity results (in m³/kg)

| Test sample | 25° C. | 37° C. |
|---|---|---|
| 0.1M sodium | 390.277 | 408.988 |
| thiosulfate in | | |
| 0.25% hyaluronic | | |
| acid gel | | |
| 0.1M sodium | 281.711 | 301.715 |
| thiosulfate in | | |
| 0.5% hyaluronic | | |
| acid gel | | |
| 0.1M sodium | 193.329 | 202.384 |
| thiosulfate in | | |
| 0.75% hyaluronic | | |
| acid gel | | |

The Table shows that decreased intrinsic viscosity is obtained with increased percentage formulation. The overall contribution to the viscosity of the dissolved hyaluronic acid decreases with increasing concentration. The intrinsic viscosity data determined at 37 degrees Celsius are marginally higher than the data determined for 25 degrees Celsius.

### REFERENCES

Brock P R, Maibach R, Childs M, et al. Sodium Thiosulfate for Protection from Cisplatin-Induced Hearing Loss. N Engl J Med 2018; 378(25): 2376-85.
Freyer D R, Chen L, Krailo M D, et al. Effects of sodium thiosulfate versus observation on development of cisplatin-induced hearing loss in children with cancer (ACCL0431): a multicentre, randomised, controlled, open-label, phase 3 trial. Lancet Oncol 2017; 18(1): 63-74.
Rolland V, Meyer F, Guitton M J, et al. A randomized controlled trial to test the efficacy of trans-tympanic injections of a sodium thiosulfate gel to prevent cisplatin-induced ototoxicity in patients with head and neck cancer. J Otolaryngol Head Neck Surg 2019; 48(1): 4.

1. A method for preventing or reducing hearing loss induced by a platinum-based drug wherein:
sodium thiosulfate (STS) is administered to the middle ear of a human subject between 8 hours before and 8 hours after the platinum-based drug is administered to said human subject.

2. The method of claim 1, wherein the platinum-based drug is selected from the group consisting of cisplatin, carboplatin, oxaliplatin, nedaplatin, picoplatin, triplatin tetranitrate, satraplatin, phenanthriplatin, or a mixture thereof, preferably wherein the platinum-based drug is selected from the group consisting of cisplatin, carboplatin, oxaliplatin and nedaplatin.

3. The method of claim 1, wherein the STS is comprised in a pharmaceutically acceptable composition, preferably a gel composition.

4. The method of claim 1, wherein the STS is administered by transtympanic or intratympanic injection.

5. The method of claim 1, wherein the dosage of the platinum-based drug is 20 mg/m² or more, 40 mg/m² or more, 75 mg/m² or more, preferably 100 mg/m² or more.

6. The method of claim 1, wherein:
when the platinum-based drug is cisplatin the dosage of cisplatin administered to said patient is 20 mg/m² or more, 40 mg/m² or more, 75 mg/m² or more, preferably 100 mg/m² or more;
when the platinum-based drug is carboplatin the dosage of carboplatin administered to said patient is 200 mg/m² or more, 250 mg/m² or more, preferably 400 mg/m² or more;
when the platinum-based drug is oxaliplatin the dosage of oxaliplatin administered to said patient is 40 mg/m² or more, 60 mg/m² or more, preferably 85 mg/m² or more.

7. The method of claim 1, wherein said STS is administered between 7 hours, 6 hours, 5 hours, 4 hours, 3 hours, 2 hours, 1 hour or 0.5 hour before and 7 hours, 6 hours, 5 hours, 4 hours, 3 hours, 2 hours, 1 hour or 0.5 hour after the plating-based drug is administered to said human subject.

8. The method of claim 1, wherein STS is administered to one or, preferably, to both middle ears.

9. The method of claim 1, wherein the STS is comprised in a composition, preferably a gel composition, wherein the intrinsic viscosity of the composition is between 80% and 120% of the intrinsic viscosity of a composition consisting of an aqueous solution of 0.5% sodium hyaluronate and 0.1 M STS in phosphate buffered saline.

10. The method of claim 9, wherein the intrinsic viscosity of the pharmaceutically acceptable composition is between 100 m³/kg and 500 m³/kg, wherein the intrinsic viscosity is calculated by using viscosity data determined with a capillary viscometer at a temperature of 37 degrees Celsius, preferably the intrinsic viscosity is between 200 m³/kg and 420 m³/kg, most preferably between 275 m³/kg and 325 m³/kg.

11. The method of claim 1, wherein the STS is administered at a temperature between 15 degrees Celsius and 40 degrees Celsius, preferably between 20 degrees Celsius and 37 degrees Celsius or preferably between 35 degrees Celsius and 38 degrees Celsius.

12. The method of claim 1, wherein the STS is comprised in a composition, preferably gel composition, and wherein STS is administered to the middle ear in a volume of at least 0.05 ml, with increasing preference at least 0.1 ml, 0.2 ml, or 0.3 ml and/or with a volume of at most 5.0 ml, with increasing preference at most 4.0 ml, or 3.0 ml.

13. The method of claim 1, wherein the middle ear is vented when STS is administered and/or wherein STS is administered to the middle ear while venting the middle ear, preferably wherein venting is via an opening, preferably a grommet, present in the eardrum, and preferably wherein STS is administered to the middle ear not through said opening.

14. The method of claim 1, wherein the STS is comprised in a pharmaceutical acceptable composition, preferably a gel composition, in a concentration of between 0.025 M and 1.0 M, preferably between 0.05 M and 0.5 M, 0.05 M and 0.2 M, more preferably between 0.075 M and 0.15 M.

15. (canceled)

16. A composition, preferably a gel composition, comprising STS and/or a composition comprising a platinum-based drug for use in preventing or reducing hearing loss induced by the platinum-based drug wherein
STS is administered to the middle ear of a human subject between 8 hours before and 8 hours after the platinum-based drug is administered to said human subject.

17. A kit comprising:
a container, preferably an ampoule, a syringe and/or an injection needle, comprising a pharmaceutical acceptable composition, preferably a gel composition, comprising STS, wherein said container, preferably an ampoule, a syringe and/or an injection needle is for administration of between 0.05 and 5.0 ml of said composition to the middle ear of a human subject, preferably wherein the said container, preferably an ampoule, a syringe and/or an injection needle is for single-use; and/or
a first container, preferably an ampoule, comprising an aqueous solution comprising STS and a second container, preferably a syringe and/or an injection needle, comprising a pre-gel, preferably a pre-gel with a composition consisting of: an aqueous solution of 1.0% sodium hyaluronate.

18. (canceled)

19. (canceled)
